(19) United States
(12) Patent Application Publication
(10) Pub. No.: US 2024/0240814 A1
(43) Pub. Date: Jul. 18, 2024

(54) Aromatherapy humidifier with mist backflow and dripping effects

(71) Applicant: Shenzhen Baowu Technology Co., LTD, Shenzhen (CN)

(72) Inventor: Changchun LI, Ganzhou (CN)

(21) Appl. No.: 18/599,575

(22) Filed: Mar. 8, 2024

(30) Foreign Application Priority Data
May 14, 2023 (CN) 202321148186.6

(51) Int. Cl. F24F 6/12; A61M 21/00; A61M 21/02; F24F 6/00; F24F 11/30

(52) U.S. Cl. CPC F24F 6/12; A61M 21/02; F24F 11/30; A61M 2021/0016; A61M 2021/0044; F24F 2006/008

(57) ABSTRACT

The present disclosure provides an aromatherapy humidifier with mist backflow and dripping effects, including a humidifier main unit body, a guide cylinder connected to the humidifier main unit body, and an upper cover body arranged at an upper part of the guide cylinder; a control circuit board and an atomization assembly are arranged in the humidifier main unit body; the aromatherapy humidifier further includes a drip hole formed in the upper cover body; the guide cylinder is of an internally hollow structure; the atomization assembly is arranged at a bottom of the guide cylinder; and mist water generated by the atomization assembly reaches the upper cover body through the guide cylinder and drips downwards from the drip hole.

### TECHNICAL FIELD

The present disclosure relates to the technical field of aromatherapy humidifier products, and in particular, to an aromatherapy humidifier with mist backflow and dripping effects.

### BACKGROUND

An aromatherapy humidifier is a common product in people's daily life. It has diverse shapes and different sizes, providing consumers with more choices. Especially in recent years, structural features of a combination of an atomizing mist component and a light component have greatly improved the use experience of humidifier and aromatherapy diffuser type products.

The invention patent No. CN202310080449.2, entitled Aromatherapy Diffuser with Raindrop Effect, specifically discloses an aromatherapy diffuser with a raindrop effect, which includes a bottom shell, a water tank arranged in the bottom shell, and an atomization module arranged on the water tank. A cloud-shaped shell is arranged above the water tank, and the water tank is provided with a water pump module for extracting liquid inside the water tank into the cloud-shaped shell. A lower end of the cloud-shaped shell is provided with a drainage hole, and a light source that can emit light with different colors is also arranged inside the cloud-shaped shell. During working, the water pump module extracts the water from the water tank into the cloud-shaped shell. The water in the cloud-shaped shell will be discharged downwards from the drainage hole and forms raindrops that fall into the water tank. The water is then extracted into the cloud-shaped shell by the water pump module, thus forming a cycle to simulate a scene of raining from the cloud to form raindrops. Meanwhile, the light source in the cloud-shaped shell can emit the light with different colors, which simulates a scene that the cloud changes its color. Furthermore, mist generated by the atomization module floats upwards to the lower end or a periphery of the cloud-shaped shell to achieve an effect of simulating a rainbow under the action of the light.

The above aromatherapy diffuser product uses a water pump to pump water to a high position, and the water then drips down to ultimately achieve a rainbow-like atomization effect. However, the structure of the product in the existing technology mentioned above not only increases the cost of the product due to the water pump module, but also is not conducive to maintenance.

Based on this, using what kind of structure to reduce the cost of the product without affecting the use experience is a problem that those skilled in the art consider.

### SUMMARY

Problems to be solved in the prior art by the present disclosure are as follows:

An aromatherapy diffuser product in the existing technology uses a water pump to pump water to a high position, and the water then drips down to ultimately achieve a rainbow-like atomization effect. However, the structure of the product in the existing technology mentioned above not only increases the cost of the product due to the water pump module, but also is not conducive to maintenance. Furthermore, the structural constitution is complicated.

Solutions of the present disclosure for solving the technical problems are as follows:

An aromatherapy humidifier with mist backflow and dripping effects is provided, including a humidifier main unit body, a guide cylinder connected to the humidifier main unit body, and an upper cover body arranged at an upper part of the guide cylinder, wherein a control circuit board and an atomization assembly are arranged in the humidifier main unit body; the aromatherapy humidifier further includes a drip hole formed in the upper cover body;
the guide cylinder is of an internally hollow structure; the atomization assembly is arranged at a bottom of the guide cylinder; mist water generated by the atomization assembly reaches the upper cover body through the guide cylinder and drips downwards from the drip hole;
a water storage tank for storing water is arranged in the humidifier main unit body; the water dripping from the drip hole flows into the water storage tank; and
the atomization assembly is electrically connected to the control circuit board.

Preferably, a mist overflow hole for overflowing mist is further formed in the upper cover body; and a mist overflow column matched with and corresponding to the mist overflow hole is integrally formed or connected inside the upper cover body.

Preferably, an atomization collection cylinder for increasing a mist spraying and water spraying height of the atomization assembly is further arranged on an inner side of a connection part between the bottom of the guide cylinder and the humidifier main unit body; and the atomization gathering cylinder is located at an upper part of the atomization assembly.

Preferably, a light-emitting diode (LED) lamp panel for generating a light effect is also arranged on an inner side of the guide cylinder; the aromatherapy humidifier further includes a waterproof lampshade arranged on an outer side of the LED lamp panel;
a bottom of the waterproof lampshade is connected to the humidifier main unit body; and a top of the waterproof lampshade and a top of the LED lamp panel extend into the upper cover body.

Preferably, a water storage slot for storing mist water is also formed in an inner side of the upper cover body; the drip hole is arranged around an outer edge of the water storage slot or is densely formed in the water storage slot; and
a water mist inlet hole communicated to a top of the guide cylinder is formed in the upper cover body.

Preferably, the humidifier main unit body includes a lower shell and a water tank body matched with and connected to an upper part of the lower shell; and the control circuit board and the atomization assembly are located in an internal space enclosed by a bottom of the water tank body and the lower shell.

Preferably, a decorative cover for decoration is also arranged on an upper part of the water tank body;
the decorative cover is provided with a guide cylinder through hole for penetrating through the guide cylinder; and
the decorative cover is further provided with a backflow hole used for making water drops dripping from the drip hole back flow and communicated to the guide cylinder.

Preferably, a lampshade connecting column for being connected to the bottom of the waterproof lampshade is arranged in the humidifier main unit body; the bottom of the waterproof lampshade is inserted into the lampshade connecting column or is connected to the lampshade connecting column by clamping or through a screw.

Preferably, the atomization gathering cylinder is connected to the humidifier main unit body through a screw; and
the atomization gathering cylinder includes a connecting part connected to the humidifier main unit body and a gathering cylinder part corresponding to the atomization assembly.

Preferably, the upper cover body includes a cover body lower part and a cover body upper part matched with and connected to the cover body lower part;
the drip hole and the mist overflow hole are both formed in the cover body lower part;
the guide cylinder and/or the upper cover body are of transparent or semi-transparent structures; and
the aromatherapy humidifier further includes a control button arranged on an outer side of the humidifier main unit body.

Technical effects achieved by solving the technical problems by the present disclosure are as follows:

Compared with the existing technology, the aromatherapy humidifier with mist backflow and dripping effects of the present disclosure includes a humidifier main unit body, a guide cylinder 17 connected to the humidifier main unit body, and an upper cover body arranged at an upper part of the guide cylinder 17; a control circuit board 111 and an atomization assembly 12 are arranged in the humidifier main unit body; the aromatherapy humidifier further includes a drip hole 184 formed in the upper cover body; the guide cylinder 17 is of an internally hollow structure; the atomization assembly 12 is arranged at a bottom of the guide cylinder 17; mist water generated by the atomization assembly 12 reaches the upper cover body through the guide cylinder 17 and drips downwards from the drip hole 184; a water storage tank for storing water is arranged in the humidifier main unit body; the water dripping from the drip hole 184 flows into the water storage tank; and the atomization assembly 12 is electrically connected to the control circuit board 111. In an actual application process, the mist generated by the atomization assembly 12 reaches the drip hole 184 of the upper cover body through the guide cylinder 17 and drips from the drip hole 184 to achieve a misty effect; and in combination with other functional components such as an illuminating effect of light, the use experience is higher. The aromatherapy humidifier of the present disclosure has a simple structure and low manufacturing cost, and is convenient to maintain.

### BRIEF DESCRIPTION OF THE DRAWINGS

FIG. 1 and FIG. 2 are schematic structural diagrams of an exploded state of an aromatherapy humidifier with mist backflow and dripping effects according to a first embodiment of the present disclosure.

FIG. 3 is a schematic structural diagram of a three-dimensional state of an aromatherapy humidifier with mist backflow and dripping effects according to a first embodiment of the present disclosure.

FIG. 4 is a schematic structural diagram of a sectional state of an aromatherapy humidifier with mist backflow and dripping effects according to a first embodiment of the present disclosure.

FIG. 5 to FIG. 7 are schematic structural diagrams of a cross-sectional state of an aromatherapy humidifier with mist backflow and dripping effects according to a first embodiment of the present disclosure.

FIG. 8 is a schematic structural diagram of a cross-sectional state of an aromatherapy humidifier with mist backflow and dripping effects according to a second embodiment of the present disclosure.

In the drawings: 11: lower shell; 12: atomization assembly; 13: waterproof lampshade; 14: water tank body; 141: lampshade connecting column; 15: atomization gathering cylinder; 16: decorative cover; 17: guide cylinder; 18: cover body lower part; 181: mist overflow column; 183: water mist inlet hole; 184: drip hole; 185: water storage slot; 19: cover body upper part; 26: decorative cover; 27: guide cylinder; 281: mist overflow column; 284: drip hole; and 286: mist overflow hole.

### DETAILED DESCRIPTION OF THE EMBODIMENTS

In order to make the objectives, technical solutions, and advantages of the present disclosure clearer, the following is a further detailed explanation of the present disclosure in conjunction with the accompanying drawings and embodiments. It should be understood that the specific embodiments described here are only intended to explain the present disclosure and are not intended to limit the present disclosure.

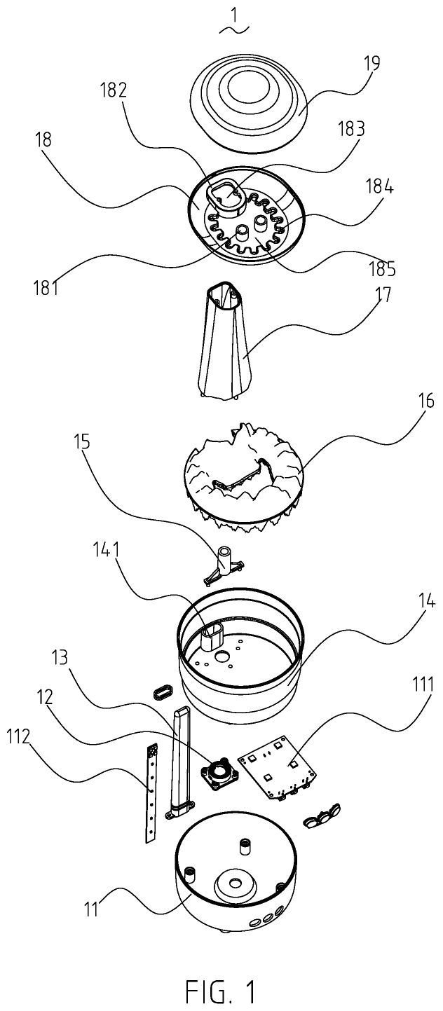

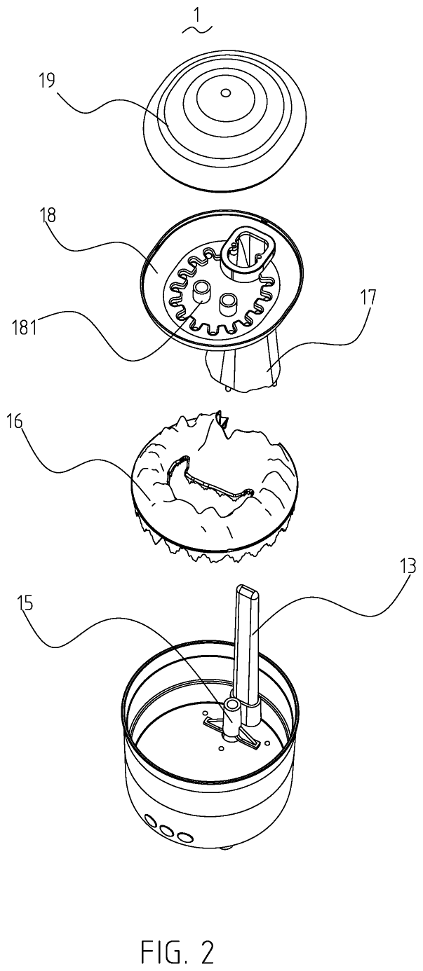

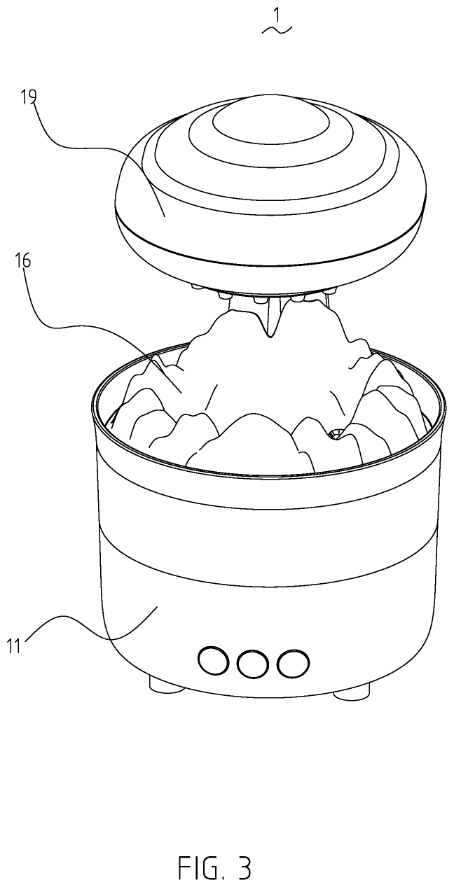

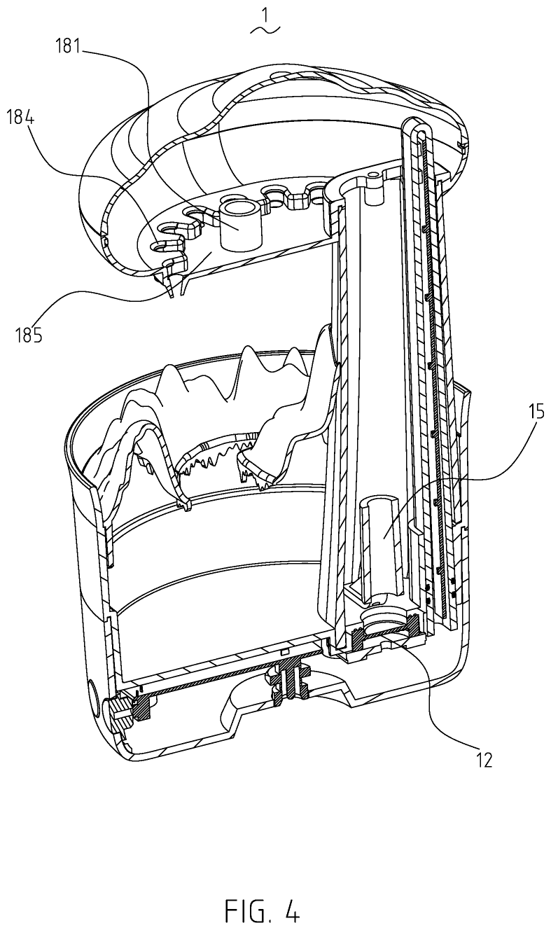

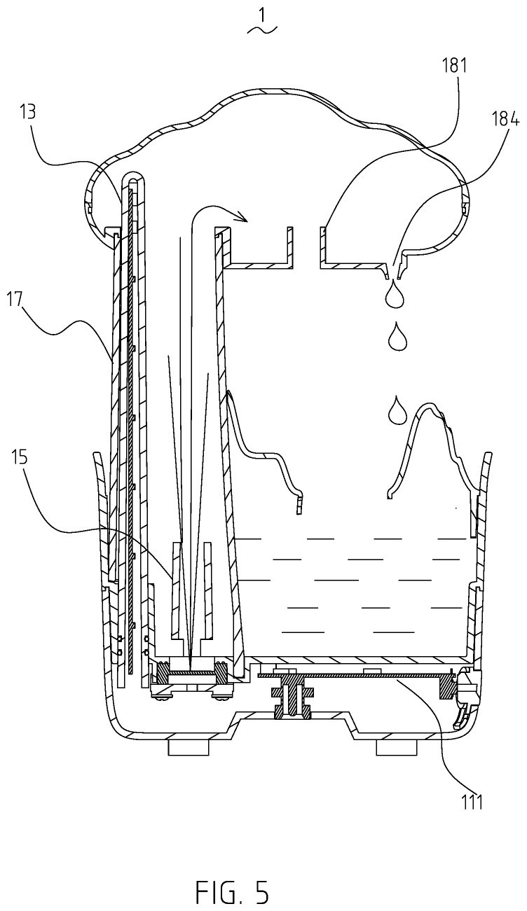

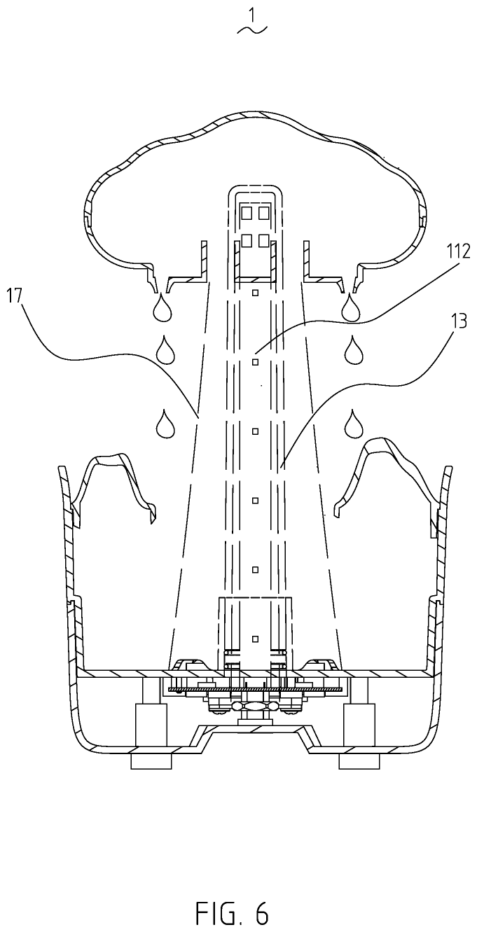

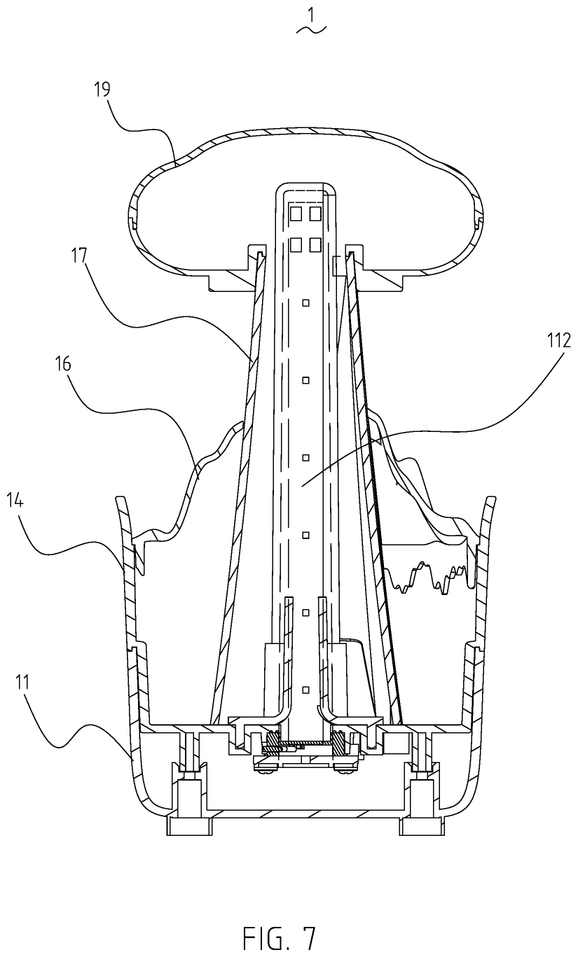

Referring to FIG. 1 to FIG. 7, an aromatherapy humidifier 1 with mist backflow and dripping effects according to a first embodiment of the present disclosure includes a humidifier main unit body, a guide cylinder 17 connected to the humidifier main unit body, and an upper cover body arranged at an upper part of the guide cylinder 17, wherein a control circuit board 111 and an atomization assembly 12 are arranged in the humidifier main unit body; and the aromatherapy humidifier further includes drip holes 184 formed in the upper cover body.

The upper cover body can be designed into a cloud-shaped structure. The present disclosure will not impose a limitation on a specific structural shape.

The guide cylinder 17 is of an internally hollow structure; the atomization assembly 12 is arranged at a bottom of the guide cylinder 17; and mist water generated by the atomization assembly 12 reaches the upper cover body through the guide cylinder 17 and drips downwards from the drip holes 184.

The upper cover body serves as a mist storage bin and a water storage bin structure.

A water storage tank for storing water is arranged in the humidifier main unit body; and the water dripping from the drip holes 184 flows into the water storage tank.

The atomization assembly 12 is electrically connected to the control circuit board 111.

The guide cylinder 17 has mist circulation and supporting effects.

In the present disclosure, the aromatherapy humidifier with the mist backflow and dripping effects of the present disclosure includes the humidifier main unit body, the guide cylinder 17 connected to the humidifier main unit body, and the upper cover body arranged at the upper part of the guide cylinder 17; the control circuit board 111 and the atomization assembly 12 are arranged in the humidifier main unit body; the aromatherapy humidifier further includes the drip holes 184 formed in the upper cover body; the guide cylinder 17 is of the internally hollow structure; the atomization assembly 12 is arranged at the bottom of the guide cylinder 17; mist water generated by the atomization assembly 12 reaches the upper cover body through the guide cylinder 17 and drips downwards from the drip holes 184; the water storage tank for storing water is arranged in the humidifier main unit body; the water dripping from the drip holes 184 flows into the water storage tank; and the atomization assembly 12 is electrically connected to the control circuit board 111. In an actual application process, the mist generated by the atomization assembly 12 reaches the drip holes 184 of the upper cover body through the guide cylinder 17 and drips from the drip holes 184 to achieve a misty effect; and in combination with other functional components such as an illuminating effect of light, the use experience is higher. The aromatherapy humidifier of the present disclosure has a simple structure and low manufacturing cost, and is convenient to maintain.

A specific principle of the present disclosure is as follows: The water mist sprayed during the working of the atomization assembly 12 is conveyed into the upper cover body, such as a cloud-shaped cover body structure; the water mist then drips back to the humidifier main unit body from the drip holes 184 of the upper cover body; and the mist atomized by the atomization assembly 12 floats outside into fog which is combined with the light effect to achieve a visual effect of cloud, rain, and fog.

In some other embodiments, a mist overflow hole for overflowing mist is further formed in the upper cover body; and a mist overflow column 181 matched with and corresponding to the mist overflow hole is integrally formed or connected inside the upper cover body.

The mist overflow hole may be formed upwards or downwards. The present disclosure will not impose a specific limitation. In this embodiment, the mist overflow hole is formed downwards, so that the mist is sprayed from the bottom.

An atomization collection cylinder 15 for increasing a mist spraying and water spraying height of the atomization assembly 12 is further arranged on an inner side of a connection part between the bottom of the guide cylinder 17 and the humidifier main unit body.

The atomization gathering cylinder 15 is located at an upper part of the atomization assembly 12.

An LED lamp panel 112 for generating a light effect is also arranged on an inner side of the guide cylinder 17; and the aromatherapy humidifier further includes a waterproof lampshade 13 arranged on an outer side of the LED lamp panel 112.

A bottom of the waterproof lampshade 13 is connected to the humidifier main unit body; and a top of the waterproof lampshade 13 and a top of the LED lamp panel 112 extend into the upper cover body.

A water storage slot 185 for storing mist water is also formed in an inner side of the upper cover body; the drip holes 184 are arranged around an outer edge of the water storage slot 185 or are densely formed in the water storage slot 185.

A water mist inlet hole 183 communicated to a top of the guide cylinder 17 is formed in the upper cover body.

A water and mist storage cavity of the upper cover body is larger than the water mist inlet hole 183 in size.

The humidifier main unit body includes a lower shell 11 and a water tank body 14, namely, the water storage tank, matched with and connected to an upper part of the lower shell 11; and the control circuit board 111 and the atomization assembly 12 are located in an internal space enclosed by a bottom of the water tank body 14 and the lower shell 11.

A decorative cover 16 for decoration is also arranged on an upper part of the water tank body 14.

The decorative cover 16 is provided with a guide cylinder through hole for penetrating through the guide cylinder 17.

The appearance of the guide cylinder 17 is matched with the guide cylinder through hole.

The decorative cover 16 is further provided with a backflow hole and/or a ventilation hole used for making water drops dripping from the drip holes 184 back flow and communicated to the guide cylinder 17. The communication mentioned here mainly means that air can flow into the guide cylinder 17 from the backflow hole or the ventilation hole, and no limitations will be imposed on structural connection relationships and on the number or positions and sizes of holes.

It should be noted that the backflow hole not only plays a role in collecting return water, but is also used for air intake. The backflow hole is communicated to the guide cylinder 17. The air flow can flow into the guide cylinder 17 through the backflow hole. Under the impact of the atomization assembly 12, the air can flow from the guide cylinder 17 into the upper cover body, namely, into the water and mist storage cavity, and is finally sprayed out from the mist overflow hole.

In an actual design, the decorative cover 16 can be integrally formed with the guide cylinder 17, and the decorative cover 16 can be taken out of the humidifier main unit body together with the guide cylinder 17.

A lampshade connecting column 141 for being connected to the bottom of the waterproof lampshade 13 is arranged in the humidifier main unit body; the bottom of the waterproof lampshade 13 is inserted into the lampshade connecting column 141 or is connected to the lampshade connecting column 141 by clamping or through a screw.

The atomization gathering cylinder 15 is connected to the humidifier main unit body through a screw.

The atomization gathering cylinder 15 includes a connecting part connected to the humidifier main unit body and a gathering cylinder part corresponding to the atomization assembly.

In a specific design, the connecting part can be small, as long as it can play a connecting role. The present disclosure will not impose a limitation on a specific shape.

The upper cover body includes a cover body lower part 18 and a cover body upper part 19 matched with and connected to the cover body lower part 18.

The drip holes 184 and the mist overflow hole are both formed in the cover body lower part 18.

The guide cylinder 17 and/or the upper cover body are of transparent or semi-transparent structures.

The aromatherapy humidifier further includes a control button arranged on an outer side of the humidifier main unit body.

A power interface can also be arranged at a bottom of the humidifier main unit body.

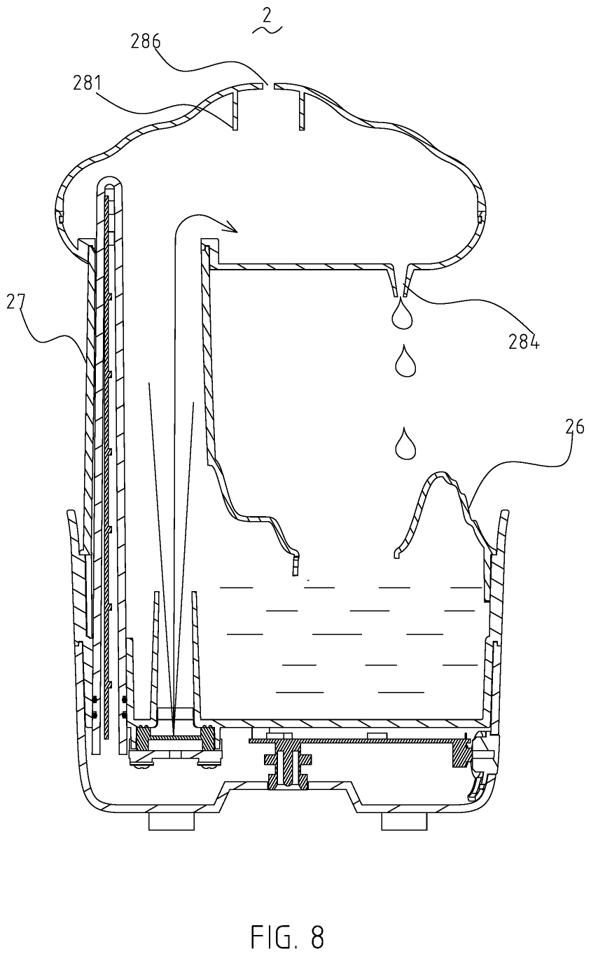

Referring to FIG. 8, in an aromatherapy humidifier 2 with mist backflow and dripping effects according to a second embodiment of the present disclosure, a mist overflow hole 286 and a mist overflow column 281 are located on an upper side of the upper cover body, so that the mist is sprayed out from the top. This is different from the first embodiment where the mist overflow column 181 is located on the lower side of the upper cover body, so that the mist is sprayed out from the bottom. The second embodiment can achieve a different spraying and dripping effect.

In the second embodiment, a decorative cover 26 and a guide cylinder 27 are integrally formed.

Compared with the existing technology, the aromatherapy humidifier 1 with the mist backflow and dripping effects of the present disclosure includes the humidifier main unit body, the guide cylinder 17 connected to the humidifier main unit body, and the upper cover body arranged at the upper part of the guide cylinder 17; the control circuit board 111 and the atomization assembly 12 are arranged in the humidifier main unit body; the aromatherapy humidifier further includes the drip holes 184 formed in the upper cover body; the guide cylinder 17 is of the internally hollow structure; the atomization assembly 12 is arranged at the bottom of the guide cylinder 17; mist water generated by the atomization assembly 12 reaches the upper cover body through the guide cylinder 17 and drips downwards from the drip holes 184; the water storage tank for storing water is arranged in the humidifier main unit body; the water dripping from the drip holes 184 flows into the water storage tank; and the atomization assembly 12 is electrically connected to the control circuit board 111. In an actual application process, the mist generated by the atomization assembly 12 reaches the drip holes 184 of the upper cover body through the guide cylinder 17 and drips from the drip holes 184 to achieve a misty effect; and in combination with other functional components such as an illuminating effect of light, the use experience is higher. The aromatherapy humidifier of the present disclosure has a simple structure and low manufacturing cost, and is convenient to maintain.

The above implementations of the present disclosure do not constitute a limitation on the scope of protection of the present disclosure. Any modification, equivalent replacement, and improvement made within the spirit and scope of the present disclosure shall fall within the scope of protection of the claims of the present disclosure.

What is claimed is:

1. An aromatherapy humidifier with mist backflow and dripping effects, comprising a humidifier main unit body, a guide cylinder connected to the humidifier main unit body, and an upper cover body arranged at an upper part of the guide cylinder, wherein a control circuit board and an atomization assembly are arranged in the humidifier main unit body; the aromatherapy humidifier further comprises a drip hole formed in the upper cover body;
the guide cylinder is of an internally hollow structure; the atomization assembly is arranged at a bottom of the guide cylinder; mist water generated by the atomization assembly reaches the upper cover body through the guide cylinder and drips downwards from the drip hole;
a water storage tank for storing water is arranged in the humidifier main unit body; the water dripping from the drip hole flows into the water storage tank; and
the atomization assembly is electrically connected to the control circuit board.

2. The aromatherapy humidifier with the mist backflow and dripping effects according to claim 1, wherein a mist overflow hole for overflowing mist is further formed in the upper cover body; and a mist overflow column matched with and corresponding to the mist overflow hole is integrally formed or connected inside the upper cover body.

3. The aromatherapy humidifier with the mist backflow and dripping effects according to claim 1, wherein an atomization gathering cylinder for increasing a mist spraying and water spraying height of the atomization assembly is further arranged on an inner side of a connection part between the bottom of the guide cylinder and the humidifier main unit body; and
the atomization gathering cylinder is located at an upper part of the atomization assembly.

4. The aromatherapy humidifier with the mist backflow and dripping effects according to claim 1, wherein a light-emitting diode (LED) lamp panel for generating a light effect is also arranged on an inner side of the guide cylinder; the aromatherapy humidifier further comprises a waterproof lampshade arranged on an outer side of the LED lamp panel;
a bottom of the waterproof lampshade is connected to the humidifier main unit body; and a top of the waterproof lampshade and a top of the LED lamp panel extend into the upper cover body.

5. The aromatherapy humidifier with the mist backflow and dripping effects according to claim 1, wherein a water storage slot for storing mist water is also formed in an inner side of the upper cover body; the drip hole is arranged around an outer edge of the water storage slot or is densely formed in the water storage slot; and
a water mist inlet hole communicated to a top of the guide cylinder is formed in the upper cover body.

6. The aromatherapy humidifier with the mist backflow and dripping effects according to claim 1, wherein the humidifier main unit body comprises a lower shell and a water tank body matched with and connected to an upper part of the lower shell; and the control circuit board and the atomization assembly are located in an internal space enclosed by a bottom of the water tank body and the lower shell.

7. The aromatherapy humidifier with the mist backflow and dripping effects according to claim 6, wherein a decorative cover for decoration is also arranged on an upper part of the water tank body;
the decorative cover is provided with a guide cylinder through hole for penetrating through the guide cylinder; and
the decorative cover is further provided with a backflow hole and/or a ventilation hole used for making water drops dripping from the drip hole back flow and communicated to the guide cylinder.

8. The aromatherapy humidifier with the mist backflow and dripping effects according to claim 4, wherein a lampshade connecting column for being connected to the bottom of the waterproof lampshade is arranged in the humidifier main unit body; the bottom of the waterproof lampshade is inserted into the lampshade connecting column or is connected to the lampshade connecting column by clamping or through a screw.

9. The aromatherapy humidifier with the mist backflow and dripping effects according to claim 3, wherein the atomization gathering cylinder is connected to the humidifier main unit body through a screw; and
the atomization gathering cylinder comprises a connecting part connected to the humidifier main unit body and a gathering cylinder part corresponding to the atomization assembly.

10. The aromatherapy humidifier with the mist backflow and dripping effects according to claim 2, wherein the upper cover body comprises a cover body lower part and a cover body upper part matched with and connected to the cover body lower part;
the drip hole and the mist overflow hole are both formed in the cover body lower part;
the guide cylinder and/or the upper cover body are of transparent or semi-transparent structures; and
the aromatherapy humidifier further comprises a control button arranged on an outer side of the humidifier main unit body.
